(19) United States
(12) Patent Application Publication
(10) Pub. No.: US 2009/0202557 A1
(43) Pub. Date: Aug. 13, 2009

(54) Compositions and methods for crystallizing antibody fragments

(76) Inventors: Maria A. Argiriadi, Wayland, MA (US); David W. Borhani, Hartsdale, NY (US); Tao Xiang, Worcester, MA (US); Chengbin Wu, Shrewsbury, MA (US); Tariq Ghayur, Holliston, MA (US)

Correspondence Address: ABBOTT BIORESEARCH, 100 RESEARCH DRIVE, WORCESTER, MA, 01605-4314, US

(21) Appl. No.: 12/322,088

(22) Filed: Jan. 29, 2009

Related U.S. Application Data
(60) Provisional application No. 61/062,887, filed on Jan. 30, 2008.
(60) Provisional application No. 61/135,739, filed on Jul. 22, 2008.

(51) Int. Cl. A61K 39/395; C07K 1/00; C07K 14/54; C07K 16/24

(52) U.S. Cl. 424/141.1; 530/387.1; 530/351; 530/388.1; 424/130.1

(57) ABSTRACT

The invention provides methods of crystallizing antibodies and fragments thereof as well as crystals produced thereby. More particularly, the invention provides methods of crystallizing human and non-human Fab fragments of antibodies, either alone or as co-crystals with their target ligand. For example, a crystal comprising a murine Fab fragment of the antibody 125-2H or a human Fab fragment of the antibody ABT-325, which bind to IL-18, are provided as well as a co-crystal of a murine Fab fragment bound to IL-18. ABT-325 and 125-2H differ significantly in combining site character and architecture, thus explaining their ability to bind IL-18 simultaneously at distinct epitopes.

### FIELD OF THE INVENTION

The present invention relates to compositions and methods for crystallizing Fab antibody fragments, and uses thereof. In particular, the invention relates to methods of crystallizing anti-interleukin 18 (IL-18) antibody Fab fragments.

### BACKGROUND OF THE INVENTION

With over 100 monoclonal antibodies currently being evaluated in clinical study phases 2 or 3, the monoclonal antibody (mAb) market is considered one of the most promising biopharmaceutical markets. Since these drugs have to be delivered to patients in single doses that often exceed 100 mg, there is an urgent need to find suitable formulations that satisfy stability, safety and patient compliance.

Highly concentrated liquid mAb formulations have a higher viscosity than less concentrated formulations, which can hinder their syringeability through more patient-friendly high gauge needles. Furthermore, the tendency of mAb molecules to aggregate exponentially increases with increased concentration, preventing compliance with safety and stability requirements. The delivery of high mAb doses therefore is restricted to large volumes, which generally have to be delivered via infusion. However, this mode of dosing is cost intensive and significantly reduces patient compliance.

For this reason, mAbs in a crystal form are desirable for use as drug substance. However few attempts have been made to evaluate this strategy due to the unpredictability associated with crystallization conditions. Although the protein insulin has been successfully crystallized, most other proteins tend to form unordered precipitates rather than crystals. Determining the crystallization conditions for a particular protein is therefore a non-trivial task. To date, there is no general rule that allows one to reliably predict a successful crystallization condition for a protein of interest.

Several screening systems are commercially available (for example, Hampton 1 and 2, Wizard I and II) that allow, on a microliter scale, screening for potentially suitable crystallization conditions for a specific protein. However, positive results obtained using such a screening system do not necessarily translate into successful crystallization at a larger, industrially applicable batch scale (see Jen et al. (2001) Pharm. Res. 18 (11):1483).

Baldock et al. ((1996) J. Crystal Growth, 168(1-4):170-174) reported on a comparison of microbatch and vapor diffusion for initial screening of crystallization conditions. Six commercially available proteins were screened using a set of crystallization solutions. The screens were performed using a common vapor diffusion method and three variants of a microbatch crystallization method. Out of 58 crystallization conditions identified, 43 (74%) were identified by microbatch, whereas 41 (71%) were identified by vapor diffusion. Twenty-six conditions were identified by both methods, and 17 (29%) would have been missed if microbatch had not been used at all. These data show that the vapor diffusion technique, which is most commonly used in initial crystallization screens, does not guarantee positive results.

Thus, the crystallization of diverse proteins cannot be carried out successfully using defined methods or algorithms. Certainly, there have been technical advances in the last 20-30 years. For example, A. McPherson provides extensive details on tactics, strategies, reagents, and devices for the crystallization of macromolecules. He does not, however, provide a method to ensure that any given macromolecule can indeed be crystallized by a skilled person with a reasonable expectation of success. McPherson states for example: “Whatever the procedure, no effort must be spared in refining and optimizing the parameters of the system, both solvent and solute, to encourage and promote specific bonding interactions between molecules and to stabilize them once they have formed. This latter aspect of the problem generally depends on the specific chemical and physical properties of the particular protein or nucleic acid being crystallized.” (McPherson (1999) Crystallization of Biological Macromolecules. Cold Spring Harbor, N.Y., Cold Spring Harbor Laboratory Press, p. 159). It is widely accepted by those skilled in the art of protein crystallization that no algorithm exists to take a new protein of interest, apply specific process steps, and thereby obtain the desired crystals.

Antibodies are particularly difficult to crystallize, due to the flexibility of the molecule. However, examples of immunoglobulin crystals do exist, such as Bence Jones proteins, which are crystals of an abnormal Ig light chain dimer (Jones (1848) Philosophical Transactions of the Royal Society, London, 138:55-62). In addition, crystals of Ig heavy chain oligomer (von Bonsdorf et al. (1938). Folia Haematologia 59:184-208) and human immunoglobulins of normal structure (two heavy chains linked to two light chains) have also been described (Putnam (1955) Science 122:275-7; Terry et al. (1968) Nature 220(164):239-41; Huber et al. (1976) Nature 264(5585):415-20; Rajan et al. (1983) Mol. Immunol. 20(7):787-99; Harris et al. (1992) Nature 360(6402): 369-72, Nisonoff et al. (1968) Cold Spring Harbor Symposia on Quant. Biol. 32:89-93; Connell et al. (1973) Canad. J. Biochem. 51(8):1137-41; Mills et al. (1983) Annals of Int. Med. 99(5):601-4; and Jentoft et al. (1982) Biochem. 21(2):289-294. For example, Margolin and co-workers reported that the therapeutic monoclonal antibody trastuzumab (Herceptin®) could be crystallized (Shenoy, et al. 2002) and that crystalline trastuzumab suspensions were therapeutically effective in a mouse tumor model, thus demonstrating retention of biological activity by crystalline trastuzumab (Yang et al. (2003) Proc. Natl. Acad. Sci. 100(12):6934-6939). However, a predictable and reliable method of forming homogeneous antibodies crystal preparations has not been described.

WO-A-02/072636 discloses the crystallization of the whole, intact antibodies Rituximab, Infliximab and Trastuzumab. Most of the crystallization experiments were performed with chemicals that have unclear toxicity, such as imidazole, 2-cyclohexyl-ethanesulfonate (CHES), methylpentanediol, copper sulphate, and 2-morpholino-ethanesulfonate (MES). Many of the examples in that application used seed crystals to initiate crystallization.

WO-A-2004/009776 discloses crystals of the anti-human TNFalpha antibody D2E7, or generically Adalimumab™, is now on the market under the trade name HUMIRA®. The application discloses crystallization experiments on a microliter scale using a sitting drop vapor diffusion technique, which involves mixing equal minute volumes (1 μl) of different crystallization buffers and D2E7 F(ab)′₂ or Fab fragments.

EP-A-0 260 610 disclosed a series of murine anti-hTNFalpha monoclonal antibodies, i.e., the neutralizing antibody AM-195, also designated MAK195, as produced by the hybridoma cell line, deposited as ECACC 87050801. An F(ab′)₂ fragment of the antibody is also known under the name Afelimomab™.

U.S. Patent Application Ser. No. 60/963,964 describes the crystallization conditions for making batch quantities of a murine anti-TNFalpha antibody F(ab′)₂ fragment (e.g., MAK-195, Abbott Laboratories).

U.S. patent application Ser. No. 11/977,677 describes the crystallization conditions for a human anti-TNFalpha antibody (e.g., Humira, Abbott Laboratories).

U.S. Patent Application Ser. No. 60/920,608 describes the crystallization conditions for a human anti-IL-12 antibody (e.g., ABT-874, Abbott Laboratories).

U.S. patent application Ser. No. 09/780,035 and 10/988,360 describe antibodies that bind to interleukin 18 (ABT-325, Abbott Laboratories), which are useful in treating a number of inflammatory diseases. However, at present, there is no technical teaching available that provides for the production of anti-IL-18 antibody, or anti-IL-18 Fab fragment, crystals. A need therefore exists for suitable crystallization conditions for providing anti-IL-18 antibody, or anti-IL-18 Fab fragment, crystals.

### SUMMARY OF THE INVENTION

The above-mentioned problems are, surprisingly, solved by the invention, which provides crystallization methods and crystals produced thereby, and their use.

In one aspect, the invention provides methods of preparing crystals of an Fab fragment of an antibody, the method comprising the steps of (a) obtaining an Fab fragment; (b) mixing the Fab fragment with a reservoir solution comprising (i) polyethylene glycol (PEG) and (ii) a buffer to make a crystallization mixture; and (c) placing the crystallization mixture on a surface until crystals form. In an embodiment the PEG is PEG400, PEG4000, or PEG6000 at a concentration in the crystallization mixture of about 5 to 20%. In an embodiment the buffer is HEPES, CAPS, Tris, cacodylate, MES, citrate, bis-tris, phosphate, CHES, MOPS, imidazole, acetate, bicine, or citrate, at a pH of about 4 to about 11. In an embodiment, the HEPES buffer is at about pH7.5. In another embodiment, the CAPS buffer is at about pH10.5. In yet another embodiment, the Tris buffer is at about pH8.5.

In an embodiment, the reservoir is selected from the group consisting of a siliconized glass slide and a sitting drop tray.

In an embodiment, the method is performed at about 0° C. to about 25° C., for example about 4° C. or about 18° C.

According to the methods of the invention, the crystallization mixture is placed on a surface for about 1 to 7 days to form crystals.

In another embodiment of the invention, the reservoir solution further comprises 2,4-methylpentanediol at a concentration of about 2 to about 40%, preferably about 5%.

In another embodiment of the invention, the reservoir solution further comprises Sulfo-Betaine 201 at a concentration of about 100 to about 500 mM.

In another embodiment of the invention, the reservoir solution further comprises MgCl₂ at a concentration of about 50 to about 500 mM, preferably about 200 mM.

The methods of the invention are useful in crystallizing Fab fragments, for example, a human or non-human Fab, such as a mouse Fab, for example, an Fab fragment of an antibody that binds to human or non-human IL-18. In an embodiment, the IL-18 is a mutant IL-18 in which all cysteine residues are mutated to alanine residues.

In another aspect, the invention provides isolated crystals of Fab fragments, for example, that bind to human or non-human IL-18. In another aspect, the invention provides an isolated co-crystal comprising an Fab fragment that is bound to human or non-human IL-18. The Fab fragment is human or non-human, such as a mouse Fab fragment of an antibody such as 125-2H or human Fab fragment of an antibody such as ABT-325. In an embodiment, the IL-18 is a mutant IL-18 in which all cysteine residues are mutated to alanine.

In an embodiment, the isolated crystal of the ABT-325 Fab fragment comprises light chain sequence SEQ ID NO:1 and heavy chain sequence SEQ ID NO:2. In an embodiment, the ABT-325 Fab fragment binds to an IL-18 amino acid sequence selected from the group consisting of SEQ ID NO:3 and SEQ ID NO:4.

In another embodiment, the ABT-325 Fab fragment binds to at least one IL-18 peptide having an amino acid sequence selected from the group consisting of Asp59-Asp76 (SEQ ID NO:7) and Glu164-Leu169 (SEQ ID NO:8), or an analog thereof with one or more amino acid substitutions, wherein the analog of IL-18 binds to antibody ABT-325. In another embodiment, the invention provides an isolated crystal comprising the Fab fragment of monoclonal antibody 125-2H, wherein the 125-2H Fab fragment comprises light chain sequence SEQ ID NO:9 and heavy chain sequence SEQ ID NO:10.

In another embodiment, the 125-2H Fab fragment binds to at least one IL-18 peptide having an amino acid sequence selected from the group consisting of Lys 176-Arg183 (SEQ ID NO:5) and Arg140-Lys148 (SEQ ID NO:6), or an analog thereof with one or more amino acid substitutions, wherein the analog binds to antibody 125-2H. In another embodiment, the invention provides methods and compositions for producing co-crystals comprising a 125-2H Fab fragment bound to human IL-18.

In another aspect, the invention provides pharmaceutical compositions comprising the isolated crystals of the invention.

### BRIEF DESCRIPTION OF THE DRAWINGS

The foregoing and other objects, features and advantages of the present invention, as well as the invention itself, will be more fully understood from the following description of preferred embodiments when read together with the accompanying drawings, in which:

FIG. 1. ABT-325 Crystals at 10× magnification.

FIG. 2. 125-2H Crystals at 10× magnification.

FIG. 3. 125-2H/IL-18 Co-Crystals at 10× magnification.

FIG. 4. IL-18 chimeras help define binding epitopes. (a) Human IL-18 and four human (N-terminus)/murine (C-terminus) chimeras. (b) Murine IL-18 and four murine (N-terminus)/human (C-terminus) chimeras. Human sequences are shown as white boxes; murine sequences are black. Residue ranges, the first mature IL-18 residue following caspase-1 cleavage (blue triangle), and epitope tags are noted for each chimera. ND: not tested.

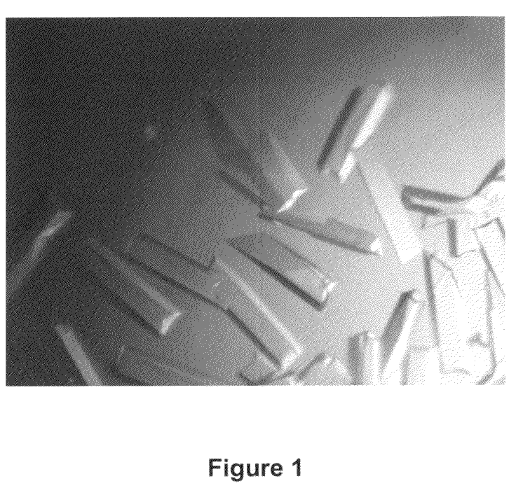

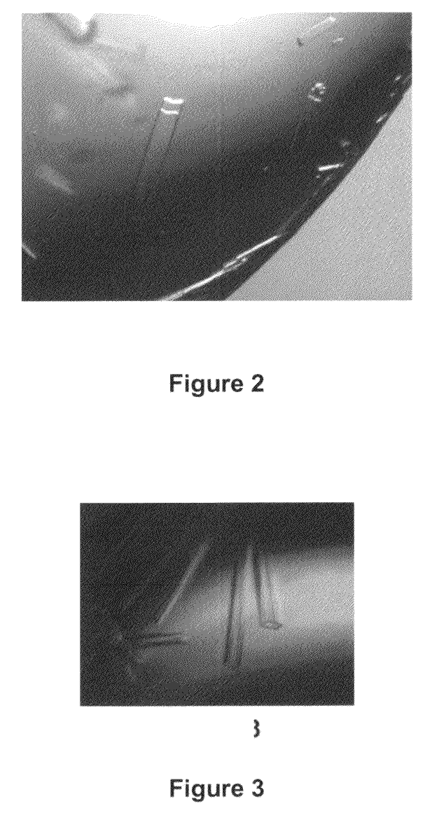

### DETAILED DESCRIPTION OF THE INVENTION

### Definitions

“Conditions enabling the formation of antibody crystals” means any condition of the solution that result in crystal formation under non-agitating conditions. This means that a solution is provided containing antibody molecules and at least one crystallization agent in concentrations sufficient to initiate crystal formation under the given conditions, such as pH and temperature of the mixture.

A “micro scale crystallization method” means any crystallization method where the volume of the crystallization mixture is between 0.1 μL and 10 μL, especially any method enabling vapor diffusion coming into effect during crystallization. For example, a method based upon vapor diffusion comprises the steps of adding a small volume of antibody solution in the microliter range with a reservoir buffer containing a crystallization agent, placing a droplet of the mixture in a sealed container adjacent to an aliquot of the reservoir buffer; allowing exchange of solvent between the droplet and the reservoir by vapor diffusion, during which the solvent content in the droplet changes and crystallization may be observed if suitable crystallization conditions are reached.

A “crystallization agent” is an agent that favors, enhances or promotes crystal formation of an antibody.

A “crystallization solution” contains a crystallization agent in dissolved form. Preferably the crystallization solution is an aqueous system, i.e., the liquid constituents thereof predominantly consist of water. For example, 80 to 100 wt.-%, or 95 to 100 wt.-%, or 98 to 100 wt.-% may be water. The term “reservoir solution” also refers to a “crystallization solution” as used for microscale crystallization by vapor diffusion techniques.

A “crystallization mixture” contains the aqueous solution of an antibody or fragment thereof and the crystallization solution or reservoir solution.

A “crystal” is one form of the solid state of matter, e.g., of a protein, which is distinct from a second solid form, i.e., the amorphous state, which exists essentially as an unorganized, heterogeneous solid. Crystals have a regular three-dimensional structure, typically referred to as a lattice. An antibody crystal comprises a regular three-dimensional array of antibody molecules. (See Giege et al., Crystallization of Nucleic Acids and Proteins, a Practical Approach, 2nd ed., pp. 1-16, Oxford University Press, New York (1999)).

A “whole” or “intact” antibody is a functional antibody that is able to recognize and bind to its antigen, as for example IL-18, in vitro and/or in vivo. The antibody may initiate subsequent immune system reactions of a patient associated with antibody-binding to its antigen, in particular direct cytotoxicity, complement-dependent cytotoxicity (CDC), and antibody-dependent cytotoxicity (ADCC). The antibody molecule typically has a structure composed of two identical heavy chains (MW each about 50 kDa) covalently bound to each other, and two identical light chains (MW each about 25 kDa), each covalently bound to one of the heavy chains. The four chains are arranged in a classic “Y” motif. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is generally composed of three CDRs and four FRs, arranged from amino-terminus to carboxy-terminus in the following order: FR1, CDR1, FR2, CDR2, FR3, CDR3, FR4. The complete antibody molecule has two antigen binding sites, i.e., is “bivalent”. The two antigen binding sites are specific for one IL-18 antigen, i.e., the antibody is “mono-specific”. The above structure may vary among different species.

“Monoclonal antibodies” are antibodies that are derived from a single clone of B lymphocytes (B cells), and recognize the same antigenic determinant. Whole monoclonal antibodies are those that have the above-mentioned classic molecular structure that includes two complete heavy chains and two complete light chains. Monoclonal antibodies are routinely produced by fusing the antibody-producing B cell with an immortal myeloma cell to generate B cell hybridomas, which continually produce monoclonal antibodies in cell culture. Other production methods are available, as for example expression of monoclonal antibodies in bacterial, yeast, insect, eukaryotic, or mammalian cell culture using phage-display technology, yeast display technology, or RNA display technology, for example; or in vivo production in genetically modified animals, such as cows, goats, pigs, rabbits, chickens, or in transgenic mice that have been modified to contain and express the entire human B cell genome; or production in genetically modified plants, such as tobacco and corn. Antibodies or fragments from all such sources may be crystallized according to this invention.

The monoclonal antibodies to be crystallized according to the invention include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass. An example of a mouse/human chimera containing variable antigen-binding portions of a murine antibody and constant portions derived from a human antibody.

“Humanized” forms of non-human (e.g., murine) antibodies are also encompassed by the invention. These are chimeric antibodies that contain minimal sequence derived from a non-human immunoglobulin. For the most part, humanized antibodies are human immunoglobulins in which residues from a complementarity determining region (CDR) or hypervariable loop (HVL) of the human immunoglobulin are replaced by residues from a CDR or HVL of a non-human species, such as mouse, rat, rabbit or nonhuman primate, having the desired functionality. Framework region (FR) residues of the human immunoglobulin may be replaced by corresponding non-human residues to improve antigen binding affinity. Furthermore, humanized antibodies may comprise residues that are found neither in the corresponding human or non-human antibody portions. These modifications may be necessary to further improve antibody efficacy.

A “human antibody” or “fully human antibody” is one that has an amino acid sequence that corresponds to that of an antibody produced by a human or that is recombinantly produced. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see, e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “neutralizing antibody”, as used herein (or an “antibody that neutralized IL-18 activity”), is intended to refer to an antibody whose binding to IL-18 results in inhibition of the biological activity of IL-18.

An “affinity matured” antibody is an antibody with one or more alterations in one or more hypervariable regions, which result in an improvement in the affinity of the antibody for antigen, compared to a parent antibody. Affinity matured antibodies have nanomolar or even picomolar affinity values for the target antigen. Affinity matured antibodies are produced by procedures known in the art. Marks et al. (1992) Bio/Technology 10:779-783 describes affinity maturation by VH and VL domain shuffling. Random mutagenesis of CDR and/or framework residues is described in Barbas et al. (1994) Proc. Nat. Acad. Sci. USA 91:3809-3813; Scier et al. (1995) Gene 169:147-155; Yelton et al. (1995) J. Immunol. 155:1994-2004; Jackson et al. (1995) J. Immunol. 154(7):3310-9; and Hawkins et al. (1992) J. MoI Biol. 226:889-896.

An “isolated antibody”, as used herein, is intended to refer to an antibody that is substantially free of other antibodies having different antigenic specificities (e.g., an isolated antibody that specifically binds IL-18 is substantially free of antibodies that specifically bind antigens other than IL-18). An isolated antibody that specifically binds IL-18 may, however, have cross-reactivity to other antigens, such as IL-18 molecules from other species. Moreover, an isolated antibody may be substantially free of other cellular material and/or chemicals.

A “functional equivalent” of a specific “parent” antibody as crystallized according to the invention is one that shows the same antigen-specificity, but differs with respect to the molecular composition of the “parent” antibody on the amino acid level or glycosylation level. The differences, however, may be merely such that the crystallization conditions do not deviate from the parameter ranges as disclosed herein.

“Encapsulation” of antibody crystals refers to a formulation where the crystals are individually coated by at least one layer of a coating material. In a preferred embodiment, such coated crystals may have a sustained dissolution rate.

“Embedding” of antibody crystals refers to a formulation where the crystals, which may be encapsulated or not, are incorporated into a solid, liquid or semi-solid carrier in a disperse manner. Such embedded crystallized antibody molecules may be released or dissolved in a controlled, sustained manner from the carrier.

A “crystallization agent of the polyalkylene polyol type” is defined in more detail below.

A “polyalkylene polyol” as used according to the invention is a straight or branched chain, in particular straight chain, poly-C₂-C₆-alkylene polyol. The polyether is formed from at least one type of a polyfunctional aliphatic alcohol carrying 2 to 6, 2 to 4 and in particular 2 or 3, preferably vicinal, hydroxyl groups and having 2 to 6, in particular 2, 3 or 4 carbon atoms, preferably forming a linear carbon backbone. Non-limiting examples are ethylene-1,2-diol (glycol), propylene-1,2-diol, propylene-1,3-diol, and n-butylene-1,3-diol and n-butylene-1,4-diol. A particularly preferred diol is glycol.

The term “polyalkylene polyol” also comprises derivatives of the same. Non-limiting examples are alkyl esters and ethers, in particular monoalkyl ethers and dialkyl ethers. “Alkyl” is in particular defined as straight or branched-chain C₁-C₆-alkyl residue, in particular, methyl, ethyl, n- or i-propyl, n-, i-, sec.-oder tert.-butyl, n- or i-pentyl; and n-hexyl.

The polyalkylene polyols, in particular the polyalkylene glycols, as used according to the invention are further characterized by a wide range of molecular weights. The molecular weight range, stated as number or weight average molecular weight, typically is in the range of about 400 to about 10,000 g/mol, as for example about 1,000 to about 8,000 g/mol, or about 2,000 to about 6,000 g/mol, about 3,000 to about 6,000 g/mol or about 3,200 to about 6,000 g/mol, as for example about 3,350 to about 6,000 g/mol, about 3,350 to about 5000 g/mol, or about 3,800 to about 4,200 g/mol, in particular about 4,000 g/mol.

Particularly preferred polyalkylene polyols are polyethylene glycols (PEGs) and polypropylene glycols (PPGs) and corresponding random or block copolymers. Specific examples of suitable polyols are PEG 400, PEG 2,000; PEG 3,000; PEG 3,350; PEG 4,000; PEG 5,000; and PEG 6,000.

The polyalkylene polyol concentration, in particular the PEG concentration, in the crystallization mixture is in the range of about 5 to about 30% (w/v), as for example about 7 to about 15% (w/v) or about 9 to about 16% (w/v) or about 9 to about 14% (w/v) or about 9 to about 12% (w/v). Preferably, PEG with an average molecular weight of about 4,000 is used in a concentration in the crystallization mixture of about 9 to about 12% (w/v) in a one-step process or about 10 to about 16% (w/v) in a multi-step process.

The polyalkylene polyols of the invention may be composed of one single type of polyol or mixtures of at least two different polyols, which may be polymerized at random or may be present as block copolymers.

### Interleukin-18 (IL-18)

Interleukin (IL)-18 is a pro-inflammatory cytokine that participates in the regulation of innate and acquired immunity (Okamura et al. (1995) Nature 378:88; Nakanishi et al. (2001) Annu. Rev. Immunol. 19:423). IL-18 acts alone or in concert with IL-12 to amplify the induction of pro-inflammatory and cytotoxic mediators such as interferon (IFN)-γ. For example, in IL-18 knockout mice, levels of IFN-γ and cytotoxic T cells decrease despite the presence of IL-12. Inhibition of IL-18 activity is beneficial in several autoimmune disease animal models, for example collagen-induced arthritis (Plater-Zyberk, et al. (2001) J. Clin. Invest. 108:1825) and colitis (Siegmund et al. (2001) Am. J. Physiol. Regul. Integr. Comp. Physiol. 281:R1264). Furthermore, IL-18 expression is dramatically increased by the chronic inflammatory state extant in human autoimmune diseases such as rheumatoid arthritis (Yamamura et al. (2001) Arthritis Rheum 44:275), multiple sclerosis (Losy et al. (2001) Acta Neurol. Scand. 104:171; Karni et al. (2002) J. Neuroimmunol. 125:134), and Crohn's disease (Ludwiczek et al. (2005) Eur. Cytokine Netw. 16:27). These observations suggest that blockade of IL-18 may be a useful human therapeutic modality (Bombardieri et al. (2007) Expert Opin. Biol. Ther. 7:31).

Despite functional divergence from the IL-1 cytokine family, IL-18 shares many similarities with IL-1. First, human IL-18 is synthesized as a biologically inactive 24-kDa precursor. Like IL-1β, IL-18 is activated and secreted following caspase-1 (and possibly other proteases) cleavage that generates the mature 18-kDa polypeptide. Despite low sequence homology to IL-1β (17%), the three-dimensional structure of IL-18 closely resembles the IL-1βαβ-trefoil fold, as shown by a recent IL-18 NMR structure determination (Kato et al. (2003) Nat. Struct. Biol. 10:966). The IL-1 and IL-18 receptors are also homologous: IL-18 binds either to the IL-18Rα chain alone or to the heterodimeric IL-18Rα/β receptor complex. IL-18 binds to IL-18Rα with ˜20 nM affinity, but signaling occurs only upon formation of the high affinity (0.2 nM) IL-18Rα/IL-18/IL-18R β ternary complex (Yoshimoto et al. (1998) J. Immunol. 161:3400; Azam et al. (2003) J. Immunol. 171:6574). Surface mutational analysis has identified two sites for IL-18 binding to IL-18Rα that are similar to those observed in the IL-1β/IL-1Rα binary complex (Vigers et al. (1997) Nature 386:190), as well as one site important for binding to IL-18R β (Kato et al. (2003) Nat. Struct. Biol. 10:966).

In a recent study, the potent (0.5 nM) IL-18-neutralizing murine monoclonal antibody (mAb), 125-2H, inhibited binding of IL-18 to IL-18Rα alone but not the heterodimeric IL-18R\tilde{α}/β receptor complex, despite rendering the ternary complex with IL-18 non-functional (Wu et al. (2003) J. Immunol. 170:5571). The structural basis for the unusual properties of 125-2H are unclear; the authors suggested that conformational changes in IL-18Rα occur upon formation of the IL-18R\tilde{α}/β receptor, thereby altering the interactions with 125-2H (Wu et al. (2003) J. Immunol. 170:5571).

### Methods and Compositions

In one aspect, the invention provides crystals and crystallization conditions for human and non-human antibody Fab fragments, such as, for example, anti-IL-18 Fab fragments. In an embodiment, the invention provides crystals of the Fab fragment of a fully-human mAb, ABT-325, that binds a distinct IL-18 epitope, as confirmed by biochemical studies, produced by a hybridoma cell line. ABT-325 is entering clinical trials for a variety of autoimmune disease indications.

In another embodiment, the invention provides crystals comprising mouse anti-IL-18 antibody 125-2H Fab fragments, produced by a hybridoma cell line. In another embodiment, the invention provides crystals comprising human IL-18 bound to the anti-IL-18 125-2H Fab fragments.

In another aspect, the invention provides methods for the preparation of antibody Fab fragment crystals by providing an aqueous crystallization mixture comprising an Fab fragment and a reservoir solution comprising at least one crystallization agent under conditions that enable the formation of Fab fragment crystals. The crystallization mixtures are obtained by adding a crystallization agent in solution or as solid to the reservoir solution comprising the Fab fragment or to the crystallization solution.

In an embodiment, the Fab fragment is a fragment of an IgG antibody, such as an IgG1, IgG2, IgG3, or IgG4 antibody. The antibody fragment may be a polyclonal antibody Fab fragment or a monoclonal antibody Fab fragment, for example, of a chimeric or non-chimeric antibody, humanized antibody, dual specific antibody, dual variable domain antibody, non-glycosylated antibody, human antibody, non-human, for example, mouse antibody. In a particular embodiment, the antibody Fab fragment to be crystallized is a non-chimeric, human antibody Fab fragment optionally further processed for improving the antigen-binding, or a fragment thereof.

In another aspect, the invention provides a crystallization method for crystallizing an anti-IL-18 Fab fragment by providing an aqueous crystallization mixture comprising an Fab fragment (e.g., in dissolved form) in a reservoir solution comprising at least one polyalkylene or polyethylene polyol, such as a polyalkylene or polyethylene glycol, as a crystallization agent; and incubating the aqueous crystallization mixture until crystals of the Fab fragment are formed; wherein the polyalkylene or polyethylene glycol is provided either (a) in one step or (b) in more than one step, wherein the antibody crystals formed in a step are not removed prior to the next step.

In one embodiment of the ABT-325 crystallization method of the invention, the pH of the aqueous crystallization mixture is in the range of about pH 8.5 to about 12.0, in particular about 9 to about 11.5, or about 9.5 to about 11.0, or about 10.0 to about 10.5, for example about 7.5.

In one embodiment of the 125-2H crystallization method of the invention, the pH of the aqueous crystallization mixture is in the range of about pH 5.5 to about 9, in particular about 6 to about 8.5, or about 6.5 to about 8, or about 7.0 to about 7.5, for example about 7.5.

In one embodiment of the 125-2H/IL-18 complex crystallization method of the invention, the pH of the aqueous crystallization mixture is in the range of about pH 4.0 to about 11, about pH 6.5 to about 10.5, in particular about 7 to about 10, or about 7.5 to about 9.5, or about 8 to about 8.5, for example about 8.5.

Both the reservoir solution and the crystallization solution may be, but do not have to be, buffered. Crystallization agent concentration and buffer molarity in the original reservoir solution is usually higher than in the crystallization mixture as it is diluted when the protein solution is added. In an embodiment, the aqueous crystallization mixture may contain at least one buffer. The buffer may, for example, comprise an acetate and or a citrate component, or an alkali metal salt thereof, as for example a sodium or a potassium salt, in particular, sodium acetate and/or sodium citrate. The salt is adjusted by the addition of an acid, in particular acetic acid or citric acid, to the required pH. In an embodiment, the buffer is HEPES, MES, bicine, CAPS, or Tris, for example.

In an embodiment of the crystallization method, the buffer concentration in the aqueous crystallization mixture is about 0 to about 0.5 M, or about 0.05 to about 0.400 M, as for example about 0.075 to about 0.300 M, or about 0.200 M.

In an embodiment, the PEG has an average molecular weight in the range of about 400 to about 20,000 g/mol in the aqueous crystallization mixture. For example, the PEG and is present in the crystallization mixture at a final concentration in the range of about 2 to about 50 (w/v) of the total volume, about 5 to about 40%, about 10 to about 30%, about 15 to about 20% or example, about 5 or about 15%.

In another embodiment, at least one of the following additional crystallization conditions are met: (1) incubation is performed for about 1 hour to about 30 days, or about ½ day to about 20 days or about 1 day to about 10 days, for example about 1 day to about 5 days, or about 2 days to about 3 days; (2) incubation is performed at a temperature between about 0° C. and about +25° C., for example about 4° C. or about 18° C.; and (3) the crystallization mixture comprises an Fab fragment at a concentration in the range of about 0.5 to about 200 mg/ml, or about 1 to about 150 mg/ml or about 2 to about 100 mg/ml, for example about 3.0 to about 50 mg/ml, in particular in the range of about 5.0 to about 10 mg/ml. The protein concentration may be determined according to standard procedures for protein determination such as, for example, by measurement of the optical density at a suitable wavelength, as for example 280 nm.

In another embodiment, the methods of the invention comprise the step of drying the crystals that are produced. Suitable drying methods include evaporative drying, spray drying, lyophilization, vacuum drying, fluid bed drying, spray freeze drying, near critical drying, supercritical drying, and nitrogen gas drying.

In a further embodiment, the crystallization methods of the invention further comprise the step of exchanging the crystallization mother liquor with a different liquid or buffer, e.g., a liquid or buffer containing at least one polyalkylene polyol different from that used for crystallization and with a molar mass in the range of about 300 to about 8,000 Daltons, or mixtures thereof, for example by centrifugation, diafiltration, ultrafiltration or other commonly used buffer exchange techniques.

In a preferred embodiment, ABT-325 Fab crystals were formed by incubation of 2 μL (˜20 mg/mL) thawed on ice mixed with 2 μL of a reservoir solution consisting of 25-30% polyethylene glycol (PEG) 400, 100 mM CAPS, pH 10.5 and suspended over the reservoir at 4° C. Rod-like crystals appeared within one day. Crystals of the ABT-325 Fab were harvested directly from their mother liquor using a fiber loop. Crystals were then flash-cooled by plunging into liquid nitrogen and stored in a liquid nitrogen refrigerator.

In another preferred embodiment, 125-2h Fab crystals were formed by incubation of 2 μL 125-2H Fab stock (˜13 mg/mL) thawed on ice mixed with 2 μL of a reservoir solution consisting of 10% polyethylene glycol (PEG) 6000, 100 mM HEPES, pH 7.5, 5% 2,4-methylpentanediol, and suspended over the reservoir (siliconized glass cover slip) at 4° C. Rod-like crystals appeared within one day. Crystals of the 125-2H Fab were harvested in mother liquor+20% propylene glycol or 25% glycerol respectively. Crystals were then flash-cooled by plunging into liquid nitrogen and stored in a liquid nitrogen refrigerator.

In another preferred embodiment, IL-18/125-2H Fab co-crystals were formed by incubation of 1.5 μL IL-18/125-2H Fab complex stock (˜20 mg/mL) thawed on ice mixed with 1.8 μL of a reservoir solution consisting of 30% PEG 4000, 100 mM Tris, pH 8.5, 0.2 M MgCl₂) and 0.3 μL of 300 mM Sulfo-Betaine 201. The mixture was suspended over the reservoir (siliconized glass cover slip) at 18° C. Rod-like crystals appeared within one week. Rod-like crystals appeared within one week. Crystals of the IL-18/125-2H Fab complex were harvested in mother liquor+20% propylene glycol or 25% glycerol respectively. Crystals were then flash-cooled by plunging into liquid nitrogen and stored in a liquid nitrogen refrigerator.

In another aspect, the invention provides crystals of an anti-IL-18 Fab fragment, and co-crystals of an anti-IL-18/IL-18 complex for example, as made by any of the methods defined herein.

In an embodiment, the crystals have the shape of needles. For example, the crystals of the invention may be characterized by a needle-like morphology with a maximum length (l) of about 2 to about 500 μm or about 100 to about 300 μm and a length/diameter (l/d) ratio of about 1 to about 100. The height of such needle-like crystals is roughly in the dimension of the diameter.

In another aspect, the invention provides pharmaceutical compositions comprising: (a) crystals of an antibody or antibody fragment prepared according to the methods defined herein; and (b) at least one pharmaceutical excipient stably maintaining the antibody crystals; wherein the composition is provided as a solid, a semisolid, or a liquid formulation. In another embodiment, the invention provides a pharmaceutical composition comprising: (a) crystals of an antibody prepared according to the methods of the invention, and (b) at least one pharmaceutical excipient, wherein the excipient embeds or encapsulates the crystals.

In another embodiment, the antibody is present in a concentration greater than about 1 mg/ml. In a particular embodiment, the antibody is present in a concentration greater than about 200 mg/ml, for example about 200 to about 600 mg/ml, or about 300 to about 500 mg/ml. In another embodiment, the pharmaceutical composition is a solid comprising about 0.1 to about 9.9% (w/w) of antibody crystals.

In an embodiment, the excipient comprises at least one polymeric biodegradable or nonbiodegradable carrier and/or at least one oil or lipid carrier, including combinations, blends, and copolymers thereof.

Exemplary polymeric carriers comprise at least one polymer selected from the group consisting of poly (acrylic acid), poly (cyanoacrylates), poly (amino acids), poly (anhydrides), poly (depsipeptide), poly (esters), poly (lactic acid), poly (lactic-co-glycolic acid) or PLGA, poly (β-hydroxybutryate), poly (caprolactone), poly (dioxanone), poly (ethylene glycol), poly (hydroxypropyl) methacrylamide, poly (organo) phosphazene, poly (ortho esters), poly (vinyl alcohol), poly (vinylpyrrolidone), maleic anhydride alkyl vinyl ether copolymers, pluronic polyols, albumin, alginate, cellulose and cellulose derivatives, collagen, fibrin, gelatin, hyaluronic acid, oligosaccharides, glycaminoglycans, and sulfated polysaccharides.

Lipid carriers include fatty acids and salts of fatty acids, fatty alcohols, fatty amines, mono-, di-, and triglycerides of fatty acids, phospholipids, glycolipids, sterols and waxes and related similar substances. Waxes are further classified in natural and synthetic products. Natural materials include waxes obtained from vegetable, animal or minerals sources such as beeswax, carnauba or montanwax. Chlorinated naphthalenes and ethylenic polymers are examples of synthetic wax products.

Oil (or oily liquid) carriers include an oil (or oily liquid) such as oleaginous almond oil, corn oil, cottonseed oil, ethyl oleate, isopropyl myristate, isopropyl palmitate, mineral oil, light mineral oil, octyldodecanol, olive oil, peanut oil, persic oil, sesame oil, soybean oil, squalane, liquid triglycerides, liquid waxes, and higher alcohols.

In another aspect, the invention provides an injectable liquid composition comprising the antibody or antibody fragment crystals obtainable by the methods of the invention, wherein the antibody or antibody fragment is present at a concentration in a range of about 10 to about 400 mg/ml, or about 50 to about 300 mg/ml, for example about 200 mg/ml.

In another aspect, the invention provides a crystal slurry composition comprising the antibody or antibody fragment crystals obtainable by the method of the invention, wherein the antibody or antibody fragment is present in a concentration greater than about 100 mg/ml, for example about 150 to about 600 mg/ml, or about 200 to about 400 mg/ml.

In another aspect, the invention provides methods for treating a mammal comprising the step of administering to the mammal an effective amount of the antibody crystals or compositions obtainable by the methods of the invention. The methods for administration of crystals and compositions thereof, may comprise, but are not restricted to, administration by the parenteral route, by the oral route, by inhalation, by injection or combinations thereof.

In a particular embodiment, the invention provides a method of treating an IL-18-related disorder in a subject comprising administering a therapeutically effective amount of the antibody crystals to the subject.

In another aspect, the invention provides uses of the anti-IL-18 antibody crystals of the invention for preparing a pharmaceutical composition for treating an IL-18 related disease.

The present invention also provides IL-18 antibody fragment crystals as defined above for use in medicine.

In a preferred embodiment of the invention, antibody protein solution and crystallization solution are combined in a ratio of about 1:1. Thus, molarities of the buffering agents/crystallization agents in the original crystallization solution are about double that in the crystallization mixture.

The crystallization methods of the invention, unless otherwise indicated, are applicable to any antibody fragment, such as an Fab fragment. The antibody may be a polyclonal antibody or, preferably, a monoclonal antibody. The antibody may be a chimeric antibody, humanized antibody, human antibody, non-human antibody, as for example a mouse antibody, each in glycosylated or non-glycosylated form. The antibody may be a dual specific antibody (dsAb) or dual variable domain antibody (DVDAb), for example.

Unless otherwise stated the crystallization methods of the invention make use of technical equipment, chemicals and methodologies well known in the art. However, as explained above, the present invention is based on the surprising finding that the selection of specific crystallization conditions, in particular, the selection of specific crystallization agents, optionally further combined with specific pH conditions and/or concentration ranges of the corresponding agents (buffer, antibody, crystallization agent), allows for the first time to prepare reproducibly stable crystals of Fab fragments, which can be further processed to form an active ingredient of a superior, highly advantageous pharmaceutical composition.

The starting material for performing the crystallization method normally comprises a concentrated solution of the antibody to be crystallized. The protein concentration may, for example, be in the range of about 1 mg/ml to about 200 mg/ml. The solution may contain additives stabilizing the dissolved antibody. In an embodiment, it is advisable to remove the additives in advance. This can be achieved by performing a buffer exchange step described herein.

Preferably, the starting material for performing the crystallization methods of the invention contains the antibody in an aqueous solution, having a pH adjusted in the range of about 5.0 to about 12.0. The pH may be adjusted by means of a suitable buffer present in a final concentration of about 1 to about 500 mM, in particular about 100 mM. The solution may contain additives, as for example in a proportion of about 0.01 to about 15, or about 0.1 to about 5, or about 0.1 to about 2 wt.-% based on the total weight of the solution, such as, for example, salts, sugars, sugar alcohols, and surfactants, in order to further stabilize the solution. The excipients should preferably be selected from physiologically acceptable compounds, routinely applied in pharmaceutical preparations. As non-limiting examples there may be mentioned salts, such as NaCl; surfactants, such as polysorbate 80 (Tween 80) and polysorbate 20 (Tween 20); sugars, such as sucrose and trehalose; cryoprotectants such as ethylene glycol, glycerol, propylene glycol, and sucrose; sugar alcohols, such as mannitol and sorbitol; and buffer agents, such as phosphate-based buffer systems, such as sodium and potassium hydrogen phosphate buffers as defined above, acetate buffer, phosphate buffer, citrate buffer, HEPES buffer, CAPS buffer, TRIS buffer, MES buffer, bicine buffer, maleate buffer or succinate buffer, and histidine buffer; and amino acids, such as histidine, arginine, and glycine, for example.

The buffer exchange may be performed by means of routine methods, for example, by dialysis, diafiltration or ultrafiltration.

If necessary, the solution will be brought to standardized crystallization conditions. In particular, the temperature will be adjusted to be in the range of about 4° C. and about 37° C. If desired or advantageous, the temperature need not be kept constant, for example the temperature may be changed, and a temperature profile that provides crystals of desired shape may be applied during the crystallization process.

A crystallization solution, containing a crystallization agent in an appropriate concentration, optionally pre-conditioned in the same way as the antibody solution, is then added to the antibody solution to form a crystallization mixture.

According to a further embodiment, the crystallization methods of the present invention may also be performed such that the crystallization mixture obtained in step a) may be supplemented with a suitable amount of pre-existing antibody crystals, as for example anti-IL-18 antibody binding fragment crystals, as seed crystals in order to initiate or boost the crystallization.

The addition of the crystallization solution may be performed continuously or discontinuously optionally under gentle agitation in order to facilitate mixing of the two liquids. Preferably, the addition is performed under conditions where the protein solution is provided under agitation and the crystallization solution (or agent in its solid form) is added in a controlled manner.

The formation of the antibody crystals is initiated by applying a polyalkylene polyol as defined above, in particular a polyalkylene glycol, and preferably a polyethylene glycol (PEG), or a mixture of at least two different polyalkylene polyols as defined above as the crystallization agent. The crystallization mixture contains the agent in a concentration that is sufficient to afford a final concentration of the polyalkylene polyol in the crystallization mixture in the range of about 5 to about 30% (w/v). A concentration gradient of the polyalkylene polyol as already described above may be applied as well.

Preferably, the crystallization solution additionally contains an acidic buffer, i.e., different from that of the antibody solution, in a concentration suitable to allow the adjustment of the pH of the crystallization mixture in the range of about 4 to about 6.

After having finished the addition of the crystallization agent to the crystallization solution, the mixture may be further incubated for about 1 hour to about 1 year in order to obtain a maximum yield of antibody crystals. If appropriate, the mixture may, for example, be agitated, gently stirred, rolled or moved in a manner known in the arte. If it is desired to additionally control the crystal size, a size-controlled crystallization method based on agitation under controlled conditions (as already explained above) may be implemented into the batch crystallization method of the invention.

The crystals obtained may be separated by known methods, for example filtration or centrifugation, as for example by centrifugation at about 200 to about 20,000 rpm, preferably about 500 to about 2,000 rpm, at room temperature of about 4° C. The remaining mother liquor may be discarded or further processed, e.g., by adding additional crystallization agent.

If necessary, the isolated crystals may be washed and subsequently dried, or the mother liquor can be substituted with a different solvent system suitable for storage and for final use of the antibodies suspended therein.

Antibody crystals formed according to the present invention may vary in their shape, as already described above. For therapeutic administration, the size of the crystals will vary depending on the route of administration, for example, for subcutaneous administration the size of the crystals may be larger than for intravenous administration. The shape of the crystals may be altered by adding specific additional additives to the crystallization mixture, as has been previously described for both protein crystals and crystals of low molecular weight, organic and inorganic molecules.

If necessary, it may be verified that the crystals are in fact crystals of the antibody. Crystals of an antibody can be analyzed microscopically for birefringence. In general, crystals, unless of cubic internal symmetry, will rotate the plane of polarization of polarized light. In yet another method, crystals can be isolated, washed, resolubilized and analyzed by SDS-PAGE and, optionally, stained with a detection antibody. Optionally, the resolubilized antibody can also be tested for binding to its antigen utilizing standard assays.

Crystals obtained according to the invention may also be crosslinked to one another. Such crosslinking may enhance stability of the crystals. Methods for crosslinking crystals are described, for example, in U.S. Pat. No. 5,849,296. Crystals can be crosslinked using a bifunctional reagent such as glutaraldehyde. Once crosslinked, crystals can be lyophilized and stored for use, for example, in diagnostic or therapeutic applications.

In some cases, it may be desirable to dry the crystals. Crystals may be dried by means of inert gases, like nitrogen gas, vacuum oven drying, lyophilization, evaporation, tray drying, fluid bed drying, spray drying, vacuum drying or roller drying. Suitable methods are well known in the art.

Crystals formed according to the invention can be maintained in the original crystallization mixture, or they can be washed and combined with other substances, such as inert carriers or ingredients to form compositions or formulations comprising crystals of the invention. Such compositions or formulations can be used, for example, in therapeutic and diagnostic applications.

In a preferred embodiment, a suitable carrier or ingredient is combined with the crystals of the invention such that the crystals of the formulation are embedded or encapsulated by an excipient. Suitable carriers may be taken from the non limiting group of: poly (acrylic acid), poly (cyanoacrylates), poly (amino acids), poly (anhydrides), poly (depsipeptide), poly (esters), poly (lactic acid), poly (lactic-co-glycolic acid) or PLGA, poly (β-hydroxybutryate), poly (caprolactone), poly (dioxanone); poly (ethylene glycol), poly (hydroxypropyl) methacrylamide, poly (organo) phosphazene, poly (ortho esters), poly (vinyl alcohol), poly (vinylpyrrolidone), maleic anhydride alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polysaccharides, blends and copolymers thereof, SAIB, fatty acids and salts of fatty acids, fatty alcohols, fatty amines, mono-, di-, and triglycerides of fatty acids, phospholipids, glycolipids, sterols and waxes and related similar substances. Waxes are further classified as natural or synthetic products. Natural materials include waxes obtained from vegetable, animal or minerals sources such as beeswax, carnauba or montanwax. Chlorinated naphthalenes and ethylenic polymers are examples of synthetic wax products.

In another aspect, the invention provides compositions and formulations comprising antibody crystals in combination with at least one carrier and/or excipient. The formulations may be solid, semisolid or liquid.

Formulations of the invention are prepared, in a form suitable for storage and/or for use, by mixing the antibody having the necessary degree of purity with a physiologically acceptable additive, such as a carrier, excipient, and/or stabilizer (see, for example, Remington's Pharmaceutical Sciences, 16th Edn., Osol, A. Ed. (1980)), in the form of suspensions, or are lyophilized or dried in another manner. Optionally, further active ingredients, such as different antibodies, biomolecules, or chemically or enzymatically synthesized low-molecular weight molecules may be incorporated as well.

Acceptable additives are non-toxic to recipients at the dosages and concentrations employed. Non-limiting examples thereof include:

Acidifying agents, such as acetic acid, citric acid, fumaric acid, hydrochloric acid, malic acid, nitric acid, phosphoric acid, diluted phosphoric acid, sulfuric acid, and tartaric acid;

Aerosol propellants, such as butane, dichlorodifluoromethane, dichlorotetrafluoroethane, isobutane, propane, and trichloromonofluoromethane;

Air displacements, such as carbon dioxide and nitrogen;

Alcohol denaturants, such as methyl isobutyl ketone and sucrose octacetate;

Alkalizing agents, such as ammonia solution, ammonium carbonate, diethanolamine, diisopropanolamine, potassium hydroxide, sodium bicarbonate, sodium borate, sodium carbonate, sodium hydroxide, and trolamine;

Antifoaming agents, such as dimethicone and simethicone;

Antimicrobial preservatives, such as benzalkonium chloride, benzalkonium chloride solution, benzelthonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, and thymol;

Antioxidants, such as ascorbic acid, ascorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, and tocopherols excipient;

Buffering agents, such as acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate, and histidine;

Chelating agents, such as edetate disodium, ethylenediaminetetraacetic acid and salts, and edetic acid;

Coating agents, such as sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcystalline wax, zein, poly amino acids, other polymers such as PLGA, etc., and SAIB;

Coloring agents, such as ferric oxide;

Complexing agents, such as ethylenediaminetetraacetic acid and salts (EDTA), edetic acid, gentisic acid ethanolamide, and oxyquinoline sulphate;

Desiccants, such as calcium chloride, calcium sulfate, and silicon dioxide;

Emulsifying and/or solubilizing agents, such as acacia, cholesterol, diethanolamine (adjunct), glyceryl monostearate, lanolin alcohols, lecithin, mono- and di-glycerides, monoethanolamine (adjunct), oleic acid (adjunct), oleyl alcohol (stabilizer), poloxamer, polyoxyethylene 50 stearate, polyoxyl 35 caster oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, soritan monooleate, sorbitan monopalmitate, sorbitan monostearate, stearic acid, trolamine, and emulsifying wax;

Filtering aids, such as powdered cellulose and purified siliceous earth;

Flavors and perfumes, such as anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, and vanillin;

Glidant and/or anticaking agents, such as calcium silicate, magnesium silicate, colloidal silicon dioxide, and talc;

Humectants, such as glycerin, hexylene glycol, propylene glycol, and sorbitol;

Ointment bases, such as lanolin, anhydrous lanolin, hydrophilic ointment, white ointment, yellow ointment, polyethylene glycol ointment, petrolatum, hydrophilic petrolatum, white petrolatum, rose water ointment, and squalane;

Plasticizers, such as castor oil, lanolin, mineral oil, petrolatum, benzyl benzyl formate, chlorobutanol, diethyl pthalate, sorbitol, diacetylated monoglycerides, diethyl phthalate, glycerin, glycerol, mono- and di-acetylated monoglycerides, polyethylene glycol, propylene glycol, triacetin, triethyl citrate, and ethanol;

Polypeptides, such as low molecular weight (less than about 10 residues);

Proteins, such as serum albumin, gelatin, and immunoglobulins;

Polymer membranes, such as cellulose acetate membranes;

Solvents, such as acetone, alcohol, diluted alcohol, amylene hydrate, benzyl benzoate, butyl alcohol, carbon tetrachloride, chloroform, corn oil, cottonseed oil, ethyl acetate, glycerin, hexylene glycol, isopropyl alcohol, methyl alcohol, methylene chloride, methyl isobutyl ketone, mineral oil, peanut oil, polyethylene glycol, propylene carbonate, propylene glycol, sesame oil, water for injection, sterile water for injection, sterile water for irrigation, purified water, liquid triglycerides, liquid waxes, and higher alcohols;

Sorbents, such as powdered cellulose, charcoal, purified siliceous earth, carbon dioxide sorbents, barium hydroxide lime, and soda lime;

Stiffening agents, such as hydrogenated castor oil, cetostearyl alcohol, cetyl alcohol, cetyl esters wax, hard fat, paraffin, polyethylene excipient, stearyl alcohol, emulsifying wax, white wax, and yellow wax;

Suppository bases, such as cocoa butter, hard fat, and polyethylene glycol;

Suspending and/or viscosity-increasing agents, such as acacia, agar, alginic acid, aluminum monostearate, bentonite, purified bentonite, magma bentonite, carbomer 934p, carboxymethylcellulose calcium, carboxymethylcellulose sodium, carboxymethycellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, and tragacanth, xanthan gum;

Sweetening agents, such as aspartame, dextrates, dextrose, excipient dextrose, fructose, mannitol, saccharin, calcium saccharin, sodium saccharin, sorbitol, solution sorbitol, sucrose, compressible sugar, confectioner's sugar, and syrup;

Tablet binders, such as acacia, alginic acid, sodium carboxymethylcellulose, microcrystalline cellulose, dextrin, ethylcellulose, gelatin, liquid glucose, guar gum, hydroxypropyl methylcellulose, methycellulose, polyethylene oxide, povidone, pregelatinized starch, and syrup;

Tablet and/or capsule diluents, such as calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, and confectioner's sugar;

Tablet disintegrants, such as alginic acid, microcrystalline cellulose, croscarmellose sodium, corspovidone, polacrilin potassium, sodium starch glycolate, starch, and pregelatinized starch.

Tablet and/or capsule lubricants, such as calcium stearate, glyceryl behenate, magnesium stearate, light mineral oil, polyethylene glycol, sodium stearyl fumarate, stearic acid, purified stearic acid, talc, hydrogenated vegetable oil, and zinc stearate;

Tonicity agents, such as dextrose, glycerin, mannitol, potassium chloride, sodium chloride;

Vehicle, such as flavored and/or sweetened aromatic elixir, compound benzaldehyde elixir, iso-alcoholic elixir, peppermint water, sorbitol solution, syrup, and tolu balsam syrup;

Vehicles, such as oleaginous almond oil, corn oil, cottonseed oil, ethyl oleate, isopropyl myristate, isopropyl palmitate, mineral oil, light mineral oil, myristyl alcohol, octyldodecanol, olive oil, peanut oil, persic oil, sesame oil, soybean oil, squalane; solid carrier sugar spheres; sterile bacteriostatic water for injection, bacteriostatic sodium chloride injection, liquid triglycerides, liquid waxes, and higher alcohols;

Water repelling agents, such as cyclomethicone, dimethicone and simethicone; and

Wetting and/or solubilizing agents, such as benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaureate, sorbitan monooleate, sorbitan monopalmitate, sorbitan monostearate, and tyloxapol.

The crystals may be combined with a polymeric carrier to provide for stability and/or sustained release. Such polymers include biocompatible and biodegradable polymers. A polymeric carrier may be a single polymer type or it may be composed of a mixture of polymer types. Nonlimiting examples of polymeric carriers have already been provided above.

### Examples of Preferred Ingredients or Excipients Include

salts of amino acids such as glycine, arginine, aspartic acid, glutamic acid, lysine, asparagine, glutamine, proline, and histidine;

monosaccharides, such as glucose, fructose, galactose, mannose, arabinose, xylose, and ribose;

disaccharides, such as lactose, trehalose, maltose, and sucrose;

polysaccharides, such as maltodextrins, dextrans, starch, and glycogen;

alditols, such as mannitol, xylitol, lactitol, and sorbitol;

glucuronic acid and galacturonic acid;

cyclodextrins, such as methyl cyclodextrin, hydroxypropyl-(3-cyclodextrin);

inorganic salts, such as sodium chloride, potassium chloride, magnesium chloride, phosphates of sodium and potassium, boric acid ammonium carbonate and ammonium phosphate;

organic salts, such as acetates, citrate, ascorbate, and lactate;

emulsifying or solubilizing agents such as acacia, diethanolamine, glyceryl monostearate, lecithin, monoethanolamine, oleic acid, oleyl alcohol, poloxamer, polysorbates, sodium lauryl sulfate, stearic acid, sorbitan monolaurate, sorbitan monostearate, and other sorbitan derivatives, polyoxyl derivatives, wax, polyoxyethylene derivatives, sorbitan derivatives; and

viscosity increasing reagents such as, agar, alginic acid and its salts, guar gum, pectin, polyvinyl alcohol, polyethylene oxide, cellulose and its derivatives propylene carbonate, polyethylene glycol, hexylene glycol and tyloxapol.

Formulations described herein also comprise an effective amount of crystalline antibody. In particular, the formulations of the invention may include a “therapeutically effective amount” or a “prophylactically effective amount” of antibody crystals of the invention. A “therapeutically effective amount” refers to an amount effective, at dosages and for periods of time necessary, to achieve the desired therapeutic result. A “therapeutically effective amount” of the antibody crystals may vary according to factors such as the disease state, age, sex, and weight of the individual, and the ability of the antibody to elicit a desired response in the individual. A therapeutically effective amount is also one in which any toxic or detrimental effects of the antibody are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Suitable dosages can readily be determined using standard methodology. The antibody is suitably administered to the patient at one time or over a series of treatments. Depending on the above mentioned factors, about 1 μg/kg to about 50 mg/kg, as for example about 0.1 to about 20 mg/kg of antibody is an initial candidate dosage for administration to the patient, whether, for example, by one or more separate administrations, or by continuous infusion. A typical daily or weekly dosage might range from about 1 μg/kg to about 20 mg/kg or more, depending on the condition, the treatment is repeated until a desired suppression of disease symptoms occurs. However, other dosage regimens may be useful. In some cases, formulations comprise a concentration of antibody of at least about 1 g/L or greater when resolubilized. In other embodiments, the antibody concentration is at least about 1 g/L to about 100 g/L when resolubilized.

Crystals of an antibody, or formulations comprising such crystals, may be administered alone or as part of a pharmaceutical preparation. Crystals of the invention may be administered by oral, parenteral, pulmonary, nasal, aural, anal, dermal, ocular, intravenous, intramuscular, intraarterial, intraperitoneal, mucosal, sublingual, subcutaneous, transdermal, topical or intracranial routes, or into the buccal cavity, for example. Specific examples of administration techniques comprise pulmonary inhalation, intralesional application, needle injection, dry powder inhalation, skin electroporation, aerosol delivery, and needle-free injection technologies, including needle-free subcutaneous administration.

The IL-18-related disorder may be selected from the following list of diseases:

TABLE 1
IL-18 Associated Diseases

Acquired Immunodeficiency Disease Syndrome
Acquired Immunodeficiency Related Diseases
Acquired pernicious anaemia
Acute coronary syndromes
Acute and chronic pain (different forms of pain)
Acute Idiopathic Polyneuritis
Acute immune disease associated with organ transplantation
Acute or chronic immune disease associated with organ transplantation
Acute Inflammatory Demyelinating Polyradiculoneuropathy
Acute ischemia
Acute liver disease
Acute rheumatic fever
Acute transverse myelitis
Addison's disease
Adult (acute) respiratory distress syndrome
Adult Still's Disease
Alcoholic cirrhosis
Alcohol-induced liver injury
Allergic diseases
Allergy
Alopecia
Alopecia areata
Alzheimer's disease
Anaphylaxis
Ankylosing spondylitis
Ankylosing spondylitis associated lung disease
Anti-Phospholipid Antibody Syndrome
Aplastic anemia
Arteriosclerosis
Arthropathy
Asthma
Atheromatous disease/arteriosclerosis
Atherosclerosis
Atopic allergy
Atopic eczema
Atopic dermatitis
Atrophic autoimmune hypothyroidism
Autoimmune bullous disease
Autoimmune dermatitis
Autoimmune diabetes
Autoimmune disorder associated with Streptococcus infection
Autoimmune Enteropathy
Autoimmune haemolytic anaemia
Autoimmune hepatitis
Autoimmune hearingloss
Autoimmune Lymphoproliferative Syndrome (ALPS)
Autoimmune mediated hypoglycaemia
Autoimmune myocarditis
Autoimmune neutropenia
Autoimmune premature ovarian failure
Autoimmune thrombocytopenia (AITP)
Autoimmune thyroid disease
Autoimmune uveitis
Bronchiolitis obliterans
Behcet's disease
Blepharitis
Bronchiectasis
Bullous pemphigoid
Cachexia
Cardiovascular Disease
Catastrophic Antiphospholipid Syndrome
Celiac Disease
Cervical Spondylosis
Chlamydia
Choleosatatis
Chronic active hepatitis
Chronic eosinophilic pneumonia
Chronic fatigue syndrome
Chronic immune disease associated with organ transplantation
Chronic ischemia
Chronic liver diseases
Chronic mucocutaneous candidiasis
Cicatricial pemphigoid
Clinically isolated Syndrome (CIS) with Risk for Multiple Sclerosis
Common varied immunodeficiency (common variable
hypogammaglobulinaemia)
Connective tissue disease associated interstitial lung disease
Conjunctivitis
Coombs positive haemolytic anaemia
Childhood Onset Psychiatric Disorder
Chronic obstructive pulmonary disease (COPD)
Crohn's disease
Cryptogenic autoimmune hepatitis
Cryptogenic fibrosing alveolitis
Dacryocystitis
Depression
Dermatitis scleroderma
Dermatomyositis
Dermatomyositis/polymyositis associated lung disease
Diabetic retinopathy
Diabetes mellitus
Dilated cardiomyopathy
Discoid lupus erythematosus
Disk herniation
Disk prolaps
Disseminated intravascular coagulation
Drug-Induced hepatitis
Drug-induced interstitial lung disease
Drug induced immune hemolytic anemia
Endocarditis
Endometriosis
Endophthalmitis
Enteropathic synovitis
Episcleritis
Erythema multiforme
Erythema multiforme major
Female infertility
Fibrosis
Fibrotic lung disease
Gestational pemphigoid
Giant cell arteritis (GCA)
Glomerulonephritides
Goitrous autoimmune hypothyroidism (Hashimoto's disease)
Goodpasture's syndrome
Gouty arthritis
Graft versus host disease (GVHD)
Grave's disease
Group B streptococci (GBS) infection
Guillain-Barré Syndrome (GBS)
Haemosiderosis associated lung disease
Hay Fever
Heart failure
Hemolytic anemia
Henoch-Schoenlein purpurea
Hepatitis B
Hepatitis C
Hughes Syndrome
Huntington's chorea
Hyperthyroidism
Hypoparathyroidism
Idiopathic leucopaenia
Idiopathic thrombocytopaenia
Idiopathic Parkinson's Disease
Idiopathic interstitial pneumonia
Idiosyncratic liver disease
IgE-mediated Allergy
Immune hemolytic anemia
Inclusion Body Myositis
Infectious diseases
Infectious ocular inflammatory disease
Inflammatory bowel disease
Inflammatory demyelinating disease
Inflammatory heart disease
Inflammatory kidney disease
Insulin dependent diabetes mellitus
Interstitial pneumonitis
IPF/UIP
Iritis
Juvenile chronic arthritis
Juvenile pernicious anaemia
Juvenile rheumatoid arthritis
Kawasaki's disease
Keratitis
Keratojuntivitis sicca
Kussmaul disease or Kussmaul-Meier Disease
Landry's Paralysis
Langerhan's Cell Histiocytosis
Linear IgA disease
Livedo reticularis
Lyme arthritis
Lymphocytic infiltrative lung disease
Macular Degeneration
Male infertility idiopathic or NOS
Malignancies
Microscopic vasculitis of the kidneys
Microscopic Polyangiitis
Mixed connective tissue disease associated lung disease
Morbus Bechterev
Motor Neuron Disorders
Mucous membrane pemphigoid
Multiple sclerosis (all subtypes: primary progressive, secondary
progressive, relapsing remitting etc.)
Multiple Organ failure
Myalgic encephalitis/Royal Free Disease
Myasthenia Gravis
Myelodysplastic Syndrome
Myocardial infarction
Myocarditis
Nephrotic syndrome
Nerve Root Disorders
Neuropathy
Non-alcoholic Steatohepatitis
Non-A Non-B Hepatitis
Optic Neuritis
Organ transplant rejection
Osteoarthritis
Osteolysis
Ovarian cancer
Ovarian failure
Pancreatitis
Parasitic diseases
Parkinson's disease
Pauciarticular JRA
Pemphigoid
Pemphigus foliaceus
Pemphigus vulgaris
Peripheral artery occlusive disease (PAOD)
Peripheral vascular disease (PVD)
Peripheral artery disease (PAD)
Phacogenic uveitis
Phlebitis
Polyarteritis nodosa (or periarteritis nodosa)
Polychondritis
Polymyalgia Rheumatica
Poliosis
Polyarticular JRA
Polyendocrine Deficiency Syndrome
Polymyositis
Polyglandular deficiency type I and polyglandular deficiency type II
polymyalgia rheumatica (PMR)
Postinfectious interstitial lung disease
Post-inflammatory interstitial lung disease
Post-Pump Syndrome
Premature ovarian failure
Primary biliary cirrhosis
Primary myxoedema
Primary parkinsonism
Primary sclerosing cholangitis
Primary sclerosing hepatitis
Primary vasculitis
Prostate and rectal cancer and hematopoietic malignancies (leukemia and
lymphoma)
Prostatitis
Psoriasis
Psoriasis type 1
Psoriasis type 2
Psoriatic arthritis
Psoriatic arthropathy
Pulmonary hypertension secondary to connective tissue disease
Pulmonary manifestation of polyarteritis nodosa
Pure red cell aplasia
Primary Adrenal Insufficiency
Radiation fibrosis
Reactive arthritis
Reiter's disease
Recurrent Neuromyelitis Optica
Renal disease NOS
Restenosis
Rheumatoid arthritis
Rheumatoid arthritis associated interstitial lung disease
Rheumatic heart disease
SAPHO (synovitis, acne, pustulosis, hyperostosis, and osteitis)
Sarcoidosis
Schizophrenia
Schmidt's syndrome
Scleroderma
Secondary Amyloidosis
Shock lung
Scleritis
Sciatica
Secondary Adrenal Insufficiency
Sepsis syndrome
Septic arthritis
Septic shock
Seronegative arthopathy
Silicone associated connective tissue disease
Sjögren's disease associated lung disease
Sjörgren's syndrome
Sneddon-Wilkinson Dermatosis
Sperm autoimmunity
Spondyloarthropathy
Spondilitis ankylosans
Stevens-Johnson Syndrome (SJS)
Still's disease
Stroke
Sympathetic ophthalmia
Systemic inflammatory response syndrome
Systemic lupus erythematosus
Systemic lupus erythematosus associated lung disease
Systemic sclerosis
Systemic sclerosis associated interstitial lung disease
Takayasu's disease/arteritis
Temporal arteritis
Th2 Type and Th1 Type mediated diseases
Thyroiditis
Toxic shock syndrome
Toxoplasmic retinitis
toxic epidermal necrolysis
Transverse myelitis
TRAPS (Tumor Necrosis Factor Receptor
Type B insulin resistance with acanthosis nigricans
Type 1 allergic reaction
Type-1 autoimmune hepatitis (classical autoimmune or lupoid hepatitis)
Type-2 autoimmune hepatitis (anti-LKM antibody hepatitis)
Type II Diabetes
Ulcerative colitic arthropathy
Ulcerative colitis
Urticaria
Usual interstitial pneumonia (UIP)
Uveitis
Vasculitic diffuse lung disease
Vasculitis
Vernal conjunctivitis
Viral retinitis
Vitiligo
Vogt-Koyanagi-Harada syndrome (VKH syndrome)
Wegener's granulomatosis
Wet macular degeneration
Wound healing
Yersinia and salmonella associated arthropathy

The IL-18-related disorder may also be selected from the following list of diseases: rheumatoid spondylitis, pulmonary disorder, intestinal disorder, cardiac disorder, inflammatory bone disorders, bone resorption disease, viral hepatitis, fulminant hepatitis, coagulation disturbances, burns, reperfusion injury, keloid formation, scar tissue formation, pyrexia, periodontal disease, obesity and radiation toxicity; a spondyloarthropathy, a metabolic disorder, anemia, pain, a hepatic disorder, a skin disorder, a nail disorder, idiopathic pulmonary fibrosis (IPF), anemia, pain, a Crohn's disease-related disorder, chronic plaque psoriasis, age-related cachexia, brain edema, inflammatory brain injury, drug reactions, edema in and/or around the spinal cord, familial periodic fevers, Felty's syndrome, post-streptococcal glomerulonephritis or IgA nephropathy, loosening of prostheses, multiple myeloma, cancer, multiple organ disorder, orchitism osteolysis, including acute, chronic, and pancreatic abscess, periodontal disease, progressive renal failure, pseudogout, pyoderma gangrenosum, relapsing polychondritis, sclerosing cholangitis, stroke, thoracoabdominal aortic aneurysm repair (TAAA), symptoms related to Yellow Fever vaccination, inflammatory diseases associated with the ear, such as chronic ear inflammation or pediatric ear inflammation, and choroidal neovascularization or lupus.

### ABT-325 Antibody

ABT-325 is a recombinant human immunoglobulin G1 (IgG1) monoclonal antibody specific for human IL-18. ABT-325 binds human IL-18, thereby inhibiting the binding of IL-18 to its receptor but does not interfere with the interaction between IL-18 and the IL-18 Binding Protein (IL-18BP), a naturally occurring IL-18 inhibitor. ABT-325 was produced in transgenic mice expressing a fully human complement of immunoglobulin variable regions with human IgG2 heavy chain constant region. Heavy and light chain variable regions were isolated from the transgenic hybridoma and grafted onto human IgG1 and K constant regions using recombinant DNA technology, resulting in a fully human antibody of IgG1 κ isotype. Two residues in the heavy chain hinge/CH2 region were mutated to prevent potential Fc gamma receptor (FcγR) and complement binding. ABT-325 is produced in mammalian cell expression system and is purified by a process that includes specific viral inactivation and removal steps. During Th1 type inflammation, interferon gamma (IFNγ) is produced and IL-18 was initially identified as an inducer of IFNγ. ABT-325 effectively neutralizes human IL-18 in vivo in a human peripheral blood mononuclear cell (PBMC)/SCID mouse chimera model stimulated with S. aureus freeze-dried cells (SAC) and blocks its ability to up-regulate the production of cytokines such as IFNγ.

ABT-325 consists of two identical IgG₁ heavy chains of 450 amino acids paired with two identical light chains of 215 amino acids. The hinge region of ABT-325 was mutated to eliminate its binding to complement and the immunoglobulin gamma Fc receptors I and IIa. The heavy chain contains 11 cysteine residues and the light chain contains 5 cysteine residues. Each heavy chain contains the following four intrachain disulfide bridges: Cys22-Cys-96, Cys-148-Cys-204, Cys265-Cys-325 and Cys371-Cys429. In each antibody molecule the two heavy chains are paired and covalently linked by interchain disulfide bridges between Cys230-Cys230 and Cys233-Cys233. The light chain contains two intrachain disufide bridges: the first between cysteines in position Cyc23-Cys88, the second between cysteines in positions Cys135-Cys195. Each heavy chain is joined with one chain through disulfide bonds at CYS_{VH}244-CYS_{VH}215. The antibody protein is glycosylated at amino acid Asparagine 301 of each heavy chain.

Amino Acid Sequence of the Light Chains of the
ABT-325 Molecule

(SEQ ID NO: 1)

EIVMTQSPATLSVSPGERATLSCRASESISSNLAWYQQKPGQAPRLFIYT
ASTRATDIPARFSGSGSGTEFTLTISSLQSEDFAVYYCQQYNNWPSITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGE
Amino Acid Sequence of the Heavy Chains of the
ABT-325 Molecule

(SEQ ID NO: 2)

EVQLVQSGTEVKKPGESLKISCKGSGYTVTSYWIGWVRQMPGKGLEWMGF
IYPGDSETRYSPTFQGQVTISADKSFNTAFLQWSSLKASDTAMYYCARVG
SGWYPYTFDIWGQGTMVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKS

### 125-2H Antibody

125-2H is a neutralizing murine immunoglobulin G1 (IgG1) monoclonal antibody specific for human IL-18 (Taniguchi et al. (1997) J. Immunol. Methods 206:107). 125-2H binds human IL-18, thereby inhibiting the binding of IL-18 to its receptor but does not inhibit the heterodimeric IL-18Rα/β receptor complex. 125-2H strongly inhibits IFN-γ production induced by IL-18 production by KG-1 cells (Taniguchi et al., 1997). 125-2H is commercially available from Maine Biotechnology Services Inc. 125-2H consists of two identical IgG1 heavy chains of 437 amino acids paired with two identical light chains of 215 amino acids. The heavy chain contains 11 cysteine residues and the light chain contains 5 cysteine residues.

Amino Acid Sequence of the Light Chains of the
125-2H Molecule

(SEQ ID NO: 3)

DIQMTQSPSSLSASLGERVSLTCRASQDIGSKLYWLQQEPDGTFKRLIYA
TSSLDSGVPKRFSGSRSGSDYSLTISSLESEDFVDYYCLQYASSPYTFGG
GTKLAIKRRADAAPTVSIFPPSSEQLTSGGASVVCFLNNFYPKDINVKWK
IDGSERQNGVLNSWTDQDSKDSTYSMSSTLTLTKDEYERHNSYTCEATHK
TSTSPIVKSFNRNE
Amino Acid Sequence of the Heavy Chains of the
125-2H Molecule

(SEQ ID NO: 4)

EIQLQQSGPELVKPGASVKVSCKASGYSFTDYFIYWVKQSHGKSLEWIGD
IDPYNGDTSYNQKFRDKATLTVDQSSTTAFMHLNSLTSEDSAVYFCARGL
RFWGQGTLVTVSAAKTTPPSVYPLAPGSAAQTNSMVTLGCLVKGYFPEPV
TVTWNSGSLSSGVHTFPAVLQSDLYTLSSSVTVPSSPRPSETVTCNVAHP
ASSTKVDKKIVPRD

Practice of the invention will be still more fully understood from the following examples, which are presented herein for illustration only and should not be construed as limiting the invention in any way. Guided by the general part of the description and on the basis of his general knowledge a skilled artisan will be enabled to provide further embodiments to the invention without undue experimentation.

### EXEMPLIFICATION

### Example 1

### Protein Expression and Purification

Human IL-18. Recombinant human pro-IL-18, in which the five cysteine residues at positions 10, 74, 104, 112, and 163 were mutated to alanine (“pro-IL-18-5C→A”, hereafter simply pro-IL-18; following UniProt Entry Q14116, mature IL-18 comprises residues 37-193), was expressed with an amino-terminal (His)₆ affinity purification tag followed by a tobacco etch virus (TEV) protease cleavage peptide in E. coli BL21 cells. Expression and purification of this mutant IL-18 was greatly simplified, compared to the wildtype protein, likely due to inhibition of polymerizing oxidation of surface-exposed residues Cys-74 and Cys-104. The following procedure was carried out at 4° C. unless specified otherwise. Cells from a 1 liter culture (stored frozen at −80° C.) were thawed, resuspended in 25 mL of Buffer A (1×PBS (150 mM NaCl, 10 mM NaPO₄, pH 7.2 [NaH₂PO₄ solution in which the pH was adjusted to 7.2 using NaOH]), 1 “protease tab” (EDTA-free complete protease inhibitor; Boehringer Mannheim, Part No. 1-873-580), and 10% glycerol), sonicated on ice (six 30-s iterations, 40% duty cycle, medium output), and centrifuged (GSA rotor, 17,000 rpm, 25 minutes). A 5 mL Ni-NTA affinity column (Qiagen) was prepared by washing sequentially with H₂O (25 mL), 100 mM NiCl₂ (50 mL), H₂O (25 mL), and Buffer B (1×PBS, 10% glycerol, 10 mL). After applying the cell lysate supernatant to the column (2 mL/min flow rate), the column was washed with Buffer B+25 mM imidazole until non-specifically bound proteins were eluted (monitored by absorbance at 280 nm). Pro-IL-18 was eluted with Buffer B+100 mM imidazole. Fractions containing a protein concentration greater than 0.3 mg/mL (Coomassie protein assay; BioRad) were pooled. The pooled sample was diluted 1:2 with 50 mM Tris, pH 7.5. Caspase-1 (1 mL of caspase 1 per 36 mg of pro-IL-18; in a spectrophotometric enzymatic assay, 10111 of this ICE preparation gave a signal of 5.0 mOD/min at 405 nm in a 10 minute assay with 100 μM Ac-YVAD-pNA; (15)) was added to the pro-IL-18 and the mixture was incubated for 40 minutes at 30° C. The sample was dialyzed against Buffer C (50 mM Tris, pH 8.0, 10% glycerol, 1 mM EDTA, 1 mM DTT, 1 mM PMSF) overnight at 4° C. The mixture was centrifuged to remove precipitated protein, filtered (0.2 μm), and loaded onto a MonoQ 10/10 anion exchange column (GE Healthcare Life Sciences; previously washed with Buffer C (40 mL); 2 mL/min). The column was washed with 5-7 column volumes (Q50 mL) of Buffer C until the OD₂₈₀ returned to baseline. Mature IL-18 was eluted with linear gradient of 0-0.5 M NaCl in Buffer B (50 column volumes [˜400 mL total volume]). A major peak eluted at ˜120 mM NaCl. The sample containing IL-18 was concentrated to ˜20 mg/mL (Ultrafree-15 Biomax 10 kDa MWCO, Millipore) and frozen at −80° C. Sample purity and identity were assessed with SDS-PAGE and mass spectrophotometry.

125-2H Fab Fragment. Murine IgG 125-2H was prepared from the hybridoma cell line (Taniguchi et al., 1997) by the ascites method at Maine Biotechnology Services (Portland, Me.). Papain gel slurry (Pierce) was activated with three volumes of Buffer D (20 mM Na₂HPO₄, 10 mM EDTA, 20 mM cysteine). The mAb was concentrated from 2.1 to 20 mg/mL in 1×PBS (Ultrafree-15 Biomax 10 kDa), mixed with 50% papain gel slurry, and incubated at 37° C. for 24 hours with gentle shaking. After overnight dialysis at 4° C. against Buffer E (50 mM Tris, pH 7.0) to remove cysteine, the sample was applied to a Protein A Sepharose 4 Fast Flow affinity column (GE Healthcare Life Sciences; 25 mL; prepared by washing with Buffer E (100 mL)) at 2 mL/minute. 125-2H Fab fractions (monitored by OD₂₈₀) were collected in the flow-through. Fractions containing the 125-2H Fab at >0.3 mg/mL were pooled, dialyzed overnight against Buffer F (50 mM Tris, pH 8.25), and then applied to a MonoQ 10/10 column (pre-equilibrated with Buffer F) at 2 mL/minute. The column was washed with 3 column volumes of Buffer F followed by elution with a 0-50% gradient of Buffer F/Buffer F+500 mM NaCl. Four peaks, which corresponded to four different species of the 125-2H Fab with distinct pI values, eluted. The major first peak was collected, concentrated (Ultrafree-15 Biomax 10 kDa) to ˜20 mg/mL, and frozen at −80° C.

ABT-325 Fab Fragment. ABT-325 IgG was expressed in Chinese Hamster Ovary cells in SR-286 media. The supernatant after cell lysis was filtered through a 0.5 μm filter and loaded onto a Protein A affinity column (pre-equilibrated 1×PBS). After washing, the IgG was eluted with Buffer G (150 mM NaCl, 0.1 M NaOAc, pH 3.5). The pooled IgG was concentrated to 20 mg/ml; papain digestion and Protein A purification was performed as described for 125-2H. Fractions containing the ABT-325 Fab at >0.3 mg/mL were pooled, concentrated to ˜20 mg/mL, and frozen at −80° C.

IL-18/125-2H Fab Fragment Complex. IL-18 and the 125-2H Fab fragment were mixed in a 1:3 mass ratio and incubated for 1 hour at 4° C. After overnight dialysis against Buffer H (50 mM Tris, pH 8.0, 10% glycerol, 2.5 mM EDTA), the sample was applied to a MonoQ 10/10 column (pre-equilibrated with Buffer H) at 2 mL/minute. The column was washed with 3 column volumes of Buffer H, and the IL-18/125-2H Fab complex was eluted with a 0-40% gradient of Buffer H/Buffer H+500 mM NaCl. The complex was concentrated to ˜10 mg/mL and frozen at −80° C.

### Example 2

### Crystallization of 125-2H Fab

Frozen 125-2H Fab stock (˜13 mg/mL) was thawed on ice. The Fab (2 μL) was mixed with 2 μL of a reservoir solution consisting of 10% polyethyleneglycol (PEG) 6000, 100 mM HEPES, pH 7.5, 5% 2,4-methylpentanediol, and suspended over the reservoir (siliconized glass cover slip) at 4° C. Rod-like crystals appeared within one day. Crystals of the 125-2H Fab were harvested in mother liquor+25% glycerol. Crystals were then flash-cooled by plunging into liquid nitrogen and stored in a liquid nitrogen refrigerator.

### Example 3

### Crystallization of ABT-325 Fab

Frozen ABT-325 Fab stock (˜20 mg/mL) was thawed on ice. The Fab (2 μL) was mixed with 2 μL of a reservoir solution consisting of 25-30% polyethyleneglycol (PEG) 400, 100 mM CAPS, pH 10.5 and suspended over the reservoir at 4° C. Rod-like crystals appeared within one day. Crystals of the ABT-325 Fab were harvested directly from their mother liquor using a fiber loop. Crystals were then flash-cooled by plunging into liquid nitrogen and stored in a liquid nitrogen refrigerator.

### Example 4

### Crystallization of IL-18/125-2H Fab Complex

Frozen IL-18/125-2H Fab complex stock (˜10 mg/mL) was thawed on ice. The complex (1.5 μL) was mixed with 1.8 μL of reservoir solution (30% PEG 4000, 100 mM Tris, pH 8.5, 0.2 M MgCl₂) and 0.3 μL of 300 mM Sulfo-Betaine 201. The mixture was suspended over the reservoir (siliconized glass cover slip) at 18° C. Rod-like crystals appeared within one week. Crystals of the IL-18/125-2H Fab complex were harvested in mother liquor+20% propylene glycol. Crystals were then flash-cooled by plunging into liquid nitrogen and stored in a liquid nitrogen refrigerator.

### Example 5

### Epitope Mapping

Human/mouse and mouse/human IL-18 chimeric proteins were produced by in vitro transcription and translation in the pro-IL-18 form, with C-terminal V5 and His tags. Caspase-1 cleavage generated the mature, tagged IL-18 chimeras. Binding assays in a sandwich ELISA format were carried out by capturing the IL-18 chimeras with the test antibody followed by detection with an anti-tag antibody. Full experimental details are provided in Wu, et al. (2003) J. Immunol. 170:5571.

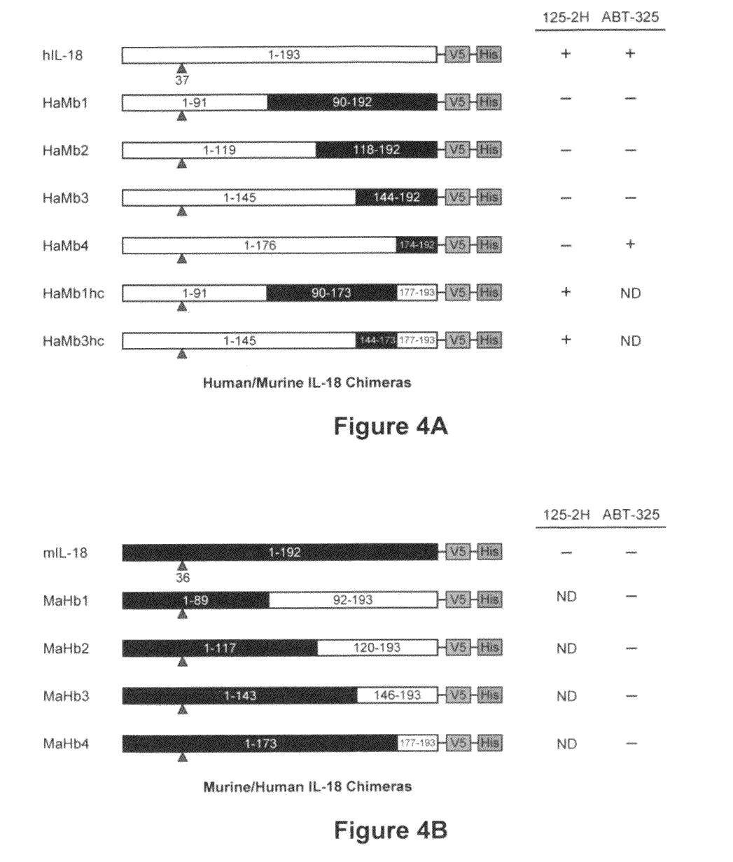

Murine IL-18 does not bind to ABT-325, and neither do chimeras in which the C-terminal human IL-18 residues 92-193, 120-193, or 146-193 were replaced by the corresponding murine sequence (FIG. 4a). But, unlike the case with 125-2H, the human (37-176)/murine (174-192) IL-18 chimera did bind to ABT-325, approximately equivalently to human IL-18. Thus, there is an important contribution to the ABT-325 epitope between residues 146-176, as only restoration of this portion restored binding. No significant binding contribution comes from residues 177-193, or binding there is due only to residues conserved between human and murine IL-18.

Four reversed IL-18 chimeras (N-terminus murine, C-terminus human; FIG. 4b) were also tested, which comprise human IL-18 residues 92-193, 120-193, 146-193, or 177-193 (Wu et al. (2003) J. Immunol. 170:5571). ABT-325 was unable to bind to any of these chimeras, indicating that an additional critical epitope lies within residues 37-91 of human IL-18.

Excluding regions that either overlap the 125-2H epitope or that are internal, IL-18 residues 146-176 contain a prominent, highly-charged surface loop, Glu164-Leu169, which is rotated approximately 90° rotated from the crystallographically-determined 125-2H epitope. Furthermore, only residues 59-76 are adjacent to this loop, surface-exposed, and are within the extreme N-terminal (37-91) segment. Thus, the chimera binding data suggest that ABT-325 binds to a conformational epitope consisting of residues 59-76 and 164-169. Engagement of this bipartite epitope by ABT-325 is consistent with simultaneous binding of ABT-325 and both 125-2H and IL-18BP to human IL-18.

Wildtype and mutant IL-18 exhibited comparable antibody binding characteristics and biological activities. Mutant IL-18 binds both 125-2H and ABT-325 with a K_{D} of ˜0.2 nM. Both ABT-325 and 125-2H neutralize recombinant (human myelomonocytic cell line KG-1 bioassay; IL-18Rα/β-driven IFN-γ production) and natural (whole blood assay; LPS+IL-12-driven IFN-γ production) human IL-18 with IC₅₀ values of 0.2 and ˜3 nM. It appears that ABT-325 binds to a more hydrophobic region of IL-18 that is distinct from the 125-2H epitope, consistent with Biacore experiments that show simultaneous binding of both antibodies to IL-18.

TABLE 2

ABT-325 and 125-2H Protein Sequences

| | | SEQ |
|---|---|---|
| | Sequence | ID |
| Protein | 12345678901234567890123456789012345678901234567890 | NO. |
| ABT-325 | EIVMTQSPATLSVSPGERATLSCRASESISSNLAWYQQKPGQAPRLFIYT | 1 |
| light | ASTRATDIPARFSGSGSGTEFTLTISSLQSEDFAVYYCQQYNNWPSITFG | |
| chain | QGTRLEIKRTVAAPSVFIFPPSDEQLKSGTASVVCLLNNFYPREAKVQWK | |
| | VDNALQSGNSQESVTEQDSKDSTYSLSSTLTLSKADYEKHKVYACEVTHQ | |
| | GLSSPVTKSFNRGE | |
| ABT-325 | EVQLVQSGTEVKKPGESLKISCKGSGYTVTSYWIGWVRQMPGKGLEWMGF | 2 |
| heavy | IYPGDSETRYSPTFQGQVTISADKSFNTAFLQWSSLKASDTAMYYCARVG | |
| chain | SGWYPYTFDIWGQGTMVTVSSASTKGPSVFPLAPSSKSTSGGTAALGCLV | |
| | KDYFPEPVTVSWNSGALTSGVHTFPAVLQSSGLYSLSSVVTVPSSSLGTQ | |
| | TYICNVNHKPSNTKVDKKVEPKS | |
| 125-2H | DIQMTQSPSSLSASLGERVSLTCRASQDIGSKLYWLQQEPDGTFKRLIYA | 3 |
| light | TSSLDSGVPKRFSGSRSGSDYSLTISSLESEDFVDYYCLQYASSPYTFGG | |
| chain | GTKLAIKRRADAAPTVSIFPPSSEQLTSGGASVVCFLNNFYPKDINVKWK | |
| | IDGSERQNGVLNSWTDQDSKDSTYSMSSTLTLTKDEYERHNSYTCEATHK | |
| | TSTSPIVKSFNRNE | |
| 125-2H | EIQLQQSGPELVKPGASVKVSCKASGYSFTDYFIYWVKQSHGKSLEWIGD | 4 |
| heavy | IDPYNGDTSYNQKFRDKATLTVDQSSTTAFMHLNSLTSEDSAVYFCARGL | |
| chain | RFWGQGTLVTVSAAKTTPPSVYPLAPGSAAQTNSMVTLGCLVKGYFPEPV | |
| | TVTWNSGSLSSGVHTFPAVLQSDLYTLSSSVTVPSSPRPSETVTCNVAHP | |
| | ASSTKVDKKIVPRD | |
| Lys176- | KEDELGDR | 5 |
| Arg183 | | |
| Arg140- | RSVPGHDNK | 6 |
| Lys148 | | |
| Asp59- | DQGNRPLFEDMTDSDCRD | 7 |
| Asp76 | | |
| Glu164- | EKERDL | 8 |
| Leu169 | | |
| IL-18 | MAAEPVEDNCINFVAMKFIDNTLYFIAEDDENLESDYFGKLESKLSVIRN | 9 |
| human | LNDQVLFIDQGNRPLFEDMTDSDCRDNAPRTIFIISMYKDSQPRGMAVTI | |
| | SVKCEKISTLSCENKIISFKEMNPPDNIKDTKSDIIFFQRSVPGHDNKMQ | |
| | FESSSYEGYFLACEKERDLFKLILKKEDELGDRSIMFTVQNED | |
| IL-18 | MAAMSEDSCVNFKEMMFIDNTLYFIPEENGDLESDNFGRLHCTTAVIRNI | 10 |
| mouse | NDQVLFVDKRQPVFEDMTDIDQSASEPQTRLIIYMYKDSEVRGLAVTLSV | |
| | KDSKMSTLSCKNKIISFEEMDPPENIDDIQSDLIFFQKRVPGHNKMEFES | |
| | SLYEGHFLACQKEDDAFKLILKKKDENGDKSVMFTLTNLHQS | |

### INCORPORATION BY REFERENCE

The contents of all cited references (including literature references, patents, patent applications, and websites) that maybe cited throughout this application are hereby expressly incorporated by reference in their entirety for any purpose. The practice of the present invention will employ, unless otherwise indicated, conventional techniques of small and large scale protein crystallization and purification, which are well known in the art.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

We claim:

1. A method of preparing crystals of an Fab fragment of an antibody, the method comprising the steps of:
(a) obtaining an Fab fragment;
(b) mixing the Fab fragment with a reservoir solution comprising polyethylene glycol and a buffer to make a crystallization mixture; and
(c) placing the crystallization mixture on a surface until crystals form.

2. The method of claim 1, wherein the polyethylene glycol is selected from the group consisting of polyethylene glycol 6000, polyethylene glycol 400, and polyethylene glycol 4000, polyethylene glycol 8000, polyethylene glycol MME 5000, and polyethylene glycol 20,000).

3. The method of claim 1, wherein the polyethylene glycol is in a concentration of about 5 to about 40%

4. The method of claim 1, wherein the buffer is selected from the group consisting of HEPES, CAPS, Tris, cacodylate, MES, citrate, bis-tris, phosphate, CHES, MOPS, imidazole, acetate, bicine, and citrate.

5. The method of claim 1, wherein the buffer is at about pH 6.5 to about pH 11.0.

6. The method of claim 4, wherein the HEPES buffer is at about pH7.5.

7. The method of claim 4, wherein the CAPS buffer is at about pH 10.5.

8. The method of claim 4, wherein the Tris buffer is at about pH8.5.

9. The method of claim 1, wherein the reservoir is selected from the group consisting of a siliconized glass slide and sitting drop wells.

10. The method of claim 1, wherein the method is performed at about 0° C. to about 25° C.

11. The method of claim 1, wherein the method is performed at about 4° C.

12. The method of claim 1, wherein the method is performed at about 18° C.

13. The method of claim 1, wherein crystallization mixture is placed on a surface for about 1 to about 7 days to form crystals.

14. The method of claim 1, wherein the reservoir solution further comprises 2,4-methylpentanediol.

15. The method of claim 13, wherein the 2,4-methylpentanediol is in a concentration of about 2 to about 10%.

16. The method of claim 1, wherein the Fab fragment is bound to IL-18.

17. The method of claim 1, further comprising MgCl₂ at a concentration of about 50 to about 500 mM

18. The method of claim 1, further comprising sulfo-betaine 201 at a concentration of about 100 to about 500 mM.

19. The method of claim 1, wherein the Fab fragment is a human Fab fragment.

20. The method of claim 1, wherein the Fab fragment is a non-human Fab fragment.

21. The method of claim 1, wherein the Fab fragment is a mouse Fab fragment.

22. The method of claim 1, wherein the Fab fragment binds a non-human IL-18.

23. The method of claim 1, wherein the Fab fragment binds a human IL-18.

24. The method of claim 1, wherein the IL-18 is a mutant IL-18 in which all cysteine residues are mutated to alanine.

25. The method of claim 1, wherein the Fab fragment comprises light chain sequence SEQ ID NO:1 and heavy chain sequence SEQ ID NO:2.

26. The method of claim 1, wherein the Fab fragment binds a protein comprising the amino acid sequence of SEQ ID No:9.

27. The method of claim 1, wherein the Fab fragment comprises light chain sequence SEQ ID NO:3 and heavy chain sequence SEQ ID NO:4.

28. The method of claim 1, wherein the Fab fragment binds a protein comprising the amino acid sequence of SEQ ID No:10.

29. An isolated crystal comprising an Fab fragment that binds to IL-18.

30. The isolated crystal of claim 29, wherein the Fab fragment is a human Fab fragment.

31. The isolated crystal of claim 29, wherein the Fab fragment is a non-human Fab fragment.

32. The isolated crystal of claim 29, wherein the Fab fragment is a mouse Fab fragment.

33. The isolated crystal of claim 29, wherein the IL-18 is human IL-18.

34. The isolated crystal of claim 29, wherein the IL-18 is non-human IL-18.

35. The isolated crystal of claim 29, wherein the Fab fragment comprises light chain sequence SEQ ID NO:1 and heavy chain sequence SEQ ID NO:2.

36. The isolated crystal of claim 29, wherein the Fab fragment binds a protein comprising the amino acid sequence of SEQ ID No:9.

37. The isolated crystal of claim 29, wherein the Fab fragment comprises light chain sequence SEQ ID NO:3 and heavy chain sequence SEQ ID NO:4.

38. The isolated crystal of claim 29, wherein the Fab fragment binds a protein comprising the amino acid sequence of SEQ ID No:10.

39. An isolated co-crystal comprising an Fab fragment that is bound to IL-18.

40. The isolated co-crystal of claim 39, wherein the Fab fragment is a human Fab fragment.

41. The isolated co-crystal of claim 39, wherein the Fab fragment is a non-human Fab fragment.

42. The isolated co-crystal of claim 41, wherein the Fab fragment is a mouse Fab fragment.

43. The isolated co-crystal of claim 39, wherein the IL-18 is human IL-18.

44. The isolated co-crystal of claim 39, wherein the IL-18 is non-human IL-18.

45. The isolated co-crystal of claim 39, wherein the Fab fragment is an Fab fragment of monoclonal antibody 125-2H.

46. The isolated crystal of claim 39, wherein the Fab fragment comprises light chain sequence SEQ ID NO:3 and heavy chain sequence SEQ ID NO:4.

47. The isolated crystal of claim 39, wherein the Fab fragment binds a protein comprising the amino acid sequence of SEQ ID No:10.

48. An isolated crystal comprising the Fab fragment of monoclonal antibody ABT-325.

49. The isolated crystal of claim 48, wherein the ABT-325 Fab fragment comprises light chain sequence SEQ ID NO:1 and heavy chain sequence SEQ ID NO:2.

50. The isolated crystal of claim 29 or 48, wherein the Fab fragment binds to at least one IL-18 peptide having an amino acid sequence selected from the group consisting of Asp59-Asp76 (SEQ ID NO:7) and Glu164-Leu169 (SEQ ID NO:8), or an analog thereof with one or more amino acid substitutions, wherein the analog binds to antibody ABT-325.

51. An isolated crystal comprising the Fab fragment of monoclonal antibody 125-2H.

52. The isolated crystal of claim 52, wherein the 125-2H Fab fragment comprises light chain sequence SEQ ID NO:3 and heavy chain sequence SEQ ID NO:4.

53. The isolated crystal of claim 52, wherein the Fab fragment binds to at least one IL-18 peptide having an amino acid sequence selected from the group consisting of Lys176-Arg183 (SEQ ID NO:5) and Arg140-Lys148 (SEQ ID NO:6), or an analog thereof with one or more amino acid substitutions, wherein the analog binds to antibody 125-2H.

54. A pharmaceutical composition comprising the isolated crystal of claim 29 or 48.

55. A method for treating a subject for a disease or a disorder by administering to the subject the crystal of claim 56 such that treatment is achieved.

56. The method of claim 58, wherein the disease is selected from the group consisting of the diseases listed in Table 1.


SEQUENCE LISTING (12 sequences)

SEQ ID NO: 1 - protein, 214 aa, Homo sapiens
EIVMTQSPATLSVSPGERATLSCRASESISSNLAWYQQKPGQAPRLFIYTASTRATDIPARFSGSGSGTEFTLTISSLQSEDFAVYYCQQYNNWPSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 2 - protein, 223 aa, Homo sapiens
EVQLVQSGTEVKKPGESLKISCKGSGYTVTSYWIGWVRQMPGKGLEWMGFIYPGDSETRYSPTFQGQVTISADKSFNTAFLQWSSLKASDTAMYYCARVGSGWYPYTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 3 - protein, 214 aa, Mus sp.
DIQMTQSPSSLSASLGERVSLTCRASQDIGSKLYWLQQEPDGTFKRLIYATSSLDSGVPKRFSGSRSGSDYSLTISSLESEDFVDYYCLQYASSPYTFGGGTKLAIKRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNE

SEQ ID NO: 4 - protein, 214 aa, Mus sp.
EIQLQQSGPELVKPGASVKVSCKASGYSFTDYFIYWVKQSHGKSLEWIGDIDPYNGDTSYNQKFRDKATLTVDQSSTTAFMHLNSLTSEDSAVYFCARGLRFWGQGTLVTVSAAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRD

SEQ ID NO: 5 - protein, 8 aa, Mus sp.
KEDELGDR

SEQ ID NO: 6 - protein, 9 aa, Mus sp.
RSVPGHDNK

SEQ ID NO: 7 - protein, 18 aa, Homo sapiens
DQGNRPLFEDMTDSDCRD

SEQ ID NO: 8 - protein, 6 aa, Homo sapiens
EKERDL

SEQ ID NO: 9 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 10 - protein, 192 aa, Mus sp.
MAAMSEDSCVNFKEMMFIDNTLYFIPEENGDLESDNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 11 - protein, 6 aa, Artificial Sequence
  Description of Artificial Sequence Synthetic 6xHis tag
HHHHHH

SEQ ID NO: 12 - protein, 4 aa, Artificial Sequence
  Description of Artificial Sequence Synthetic peptide
YVAD